(19) United States
(12) Patent Application Publication
(10) Pub. No.: US 2012/0150015 A1
(43) Pub. Date: Jun. 14, 2012

(54) SYSTEM AND METHOD FOR DETECTING HEALING ADJACENT TO IMPLANTED MEDICAL DEVICE

(75) Inventors: Gurpreet S. Sandhu, Rochester, MN (US); Martin G. Rodriguez-Porcel, Rochester, MN (US)

(73) Assignee: MAYO FOUNDATION FOR MEDICAL EDUCATION AND RESEARCH, Rochester, MN (US)

(21) Appl. No.: 13/392,193

(22) PCT Filed: Aug. 5, 2010
(86) PCT No.: PCT/US10/44513

§ 371 Date: Feb. 24, 2012

Related U.S. Application Data
(60) Provisional application No. 61/239,263, filed on Sep. 2, 2009.

(51) Int. Cl. A61B 6/00; A61B 5/05; G01N 23/04; A61B 8/12; A61B 5/055; B05D 3/10; A61B 5/00; A61B 8/00

(52) U.S. Cl. 600/410; 600/300; 600/425; 600/437; 600/476; 600/407; 427/2.12; 378/62

(57) ABSTRACT

A method of detecting healing adjacent to an implanted medical device can first include pre-treating a surface of the medical device prior to implantation, thereby defining a pre-treated surface of the medical device. Then, subsequent to implantation, a portion of the pre-treated surface that is not covered by endothelial tissue is detected by a detection device.

### CROSS-REFERENCE

The priority benefit of U.S. Provisional Application No. 61/239,263, filed Sep. 2, 2009, is hereby claimed and its entire contents are incorporated herein by reference.

### FIELD OF THE DISCLOSURE

The present disclosure generally relates to medical devices and, more particularly, to a system and method for determining the degree to which healing has occurred adjacent to an implanted medical device.

### BACKGROUND

For many years, coronary obstructions were treated solely by balloon angioplasty. Due to the risk of blood vessel narrowing, however, metal stents have been more recently introduced to treat coronary obstructions. Subsequent to implantation, metal stents become covered with endothelial tissue, which helps to prevent thrombosis. The amount of time required for an implanted stent to become completely covered by endothelial tissue differs for each patient and is further delayed in the case of drug eluting stents. Therefore, the risk of thrombosis at any given time after implantation can differ for each patient.

### SUMMARY

One aspect of the present disclosure provides a method of detecting healing adjacent to an implanted medical device, wherein the method comprises first pre-treating a surface of the medical device prior to implantation, thereby defining a pre-treated surface of the medical device. Then, the method includes detecting a portion of the pre-treated surface of the medical device subsequent to imp antation, the portion of the pre-treated surface not being covered by endothelial tissue.

In one embodiment, detecting the portion of the pre-treated surface of the medical device comprises delivering a detection device into the blood vessel to a position adjacent the implanted medical device.

In one embodiment, detecting the portion of the pre-treated surface of the medical device comprises detecting the pre-treated surface with at least one of: (a) an optical coherence tomography device, (b) an intravascular optical coherence tomography device, (c) an ultrasound device, (d) an intravascular ultrasound device, (e) a magnetic resonance imaging device, (t) an intravascular magnetic resonance imaging device, (g) an x-ray imaging device, (h) an intravascular x-ray imaging device, (i) a fluorescence imaging device, (j) an intravascular fluorescence imaging device, (k) a near-infrared fluorescence (NIRF) imaging device, (l) an intravascular near-infrared fluorescence (NIRF) imaging device, and (m) a camera.

In one embodiment, pre-treating the surface of the medical device comprises structurally treating the surface of the medical device.

In one embodiment, structurally treating the surface of the medical device comprises at least one of: (a) forming at least one indentation on the surface of the medical device, (b) forming at least one protrusion on the surface of the medical device, (c) scratching the surface of the medical device, (d) applying a design to the surface of the medical device, (e) forming a pattern on the surface of the medical device, (f) attaching a plurality of dots to the surface of the medical device, (g) attaching a plurality of quantum dots to the surface of the medical device, (h) attaching a layer of a polymer material to the surface of the medical device, (i) attaching a layer of a metallic material to the surface of the medical device, (j) attaching a layer of a polymeric material to the surface of the medical device, and (k) attaching a layer of a ceramic material to the surface of the medical device.

In one embodiment, the method further comprises generating a live image of at least the pre-treated surface of the medical device that is not covered by endothelial tissue; and comparing the live image to a reference image for determining to what degree the implanted medical device is covered by endothelial tissue.

In one embodiment, the method further comprises transmitting the live image to a processor and obtaining the reference image from a memory device in communication with the processor prior to comparing the live image to the reference image.

In one embodiment, the method further comprises determining what percentage of the implanted medical device is not covered by endothelial tissue.

In one embodiment, the method further comprises determining what percentage of the implanted medical device is covered by endothelial tissue.

Another aspect of the present disclosure includes a method of detecting healing adjacent to an implanted medical device, wherein the method first comprises applying one or more detectable agents to one or more target surfaces subsequent to implantation of the medical device. The one or more target surfaces can be selected from the group consisting of: (a) a surface of the medical device that is not covered by endothelial tissue, and (b) a surface of a layer of endothelial tissue, the layer covering at least a portion of the medical device. Then, the method includes detecting the one or more detectable agents applied to the one or more target surfaces for determining to what degree the implanted medical device is covered by endothelial tissue.

In one embodiment, applying the one or more detectable agents comprises at least one of: (a) applying a paint to the surface of the device that is not covered by endothelial tissue, (b) applying a dye to the surface of the medical device that is not covered by endothelial tissue, (c) applying a magnetic field to the medical device and attaching a plurality of magnetic particles to the surface of the device that is not covered by endothelial tissue, (d) attaching a plurality of microbubbles to the surface of the medical device that is not covered by endothelial tissue, (e) attaching a plurality of colored microbubbles to the surface of the medical device that is not covered by endothelial tissue, (f) attaching a plurality of nanoparticles to the surface of the medical device that is not covered by endothelial tissue, (g) attaching a plurality of antigens to a plurality of antibodies connected to the surface of the medical device that is not covered by endothelial tissue, (h) attaching a plurality of molecules to a molecularly imprinted polymer connected to the surface of the medical device that is not covered by endothelial tissue, (i) applying a paint to the surface of the layer of endothelial tissue that is covering at least a portion of the medical device, (j) applying a dye to the surface of the layer of endothelial tissue that is covering at least a portion of the medical device, (k) attaching a plurality of microbubbles to the surface of the layer of endothelial tissue that is covering at least a portion of the medical device, (l) attaching a plurality of colored microbubbles to the surface of the layer of endothelial tissue that is covering at least a portion of the medical device, (m) attaching a plurality of microbubbles and liposomes to the surface of the layer of endothelial tissue that is covering at least a portion of the medical device, (n) attaching a plurality of microbubbles and liposomes modified with fluorescent agents to the surface of the layer of endothelial tissue that is covering at least a portion of the medical device, and (o) attaching a plurality of nanoparticles to the surface of the layer of endothelial tissue that is covering at least a portion of the medical device.

In one embodiment, the method further comprises pre-treating a surface of the medical device prior to implantation, thereby defining a pre-treated surface of the medical device, such that applying the one or more detectable agents to the surface of the medical device that is not covered by endothelial tissue comprises applying one or more detectable agents to the pre-treated surface.

In one embodiment, the method further comprises pre-treating the surface of the medical device prior to implantation comprises at least one of: (a) attaching a molecularly imprinted polymer to the surface of the medical device, (b) attaching a plurality of antibodies to the surface of the medical device, (c) forming at least one indentation on the surface of the medical device, (d) forming at least one protrusion on the surface of the medical device, (e) scratching the surface of the medical device, (f) applying a design to the surface of the medical device, (g) forming a pattern on the surface of the medical device, (h) attaching a plurality of dots to the surface of the medical device, (i) attaching a plurality of quantum dots to the surface of the medical device, (j) attaching a layer of a polymer material to the surface of the medical device, (k) attaching a layer of a metallic material to the surface of the medical device, (l) attaching a layer of a polymeric material to the surface of the medical device, and (m) attaching a layer of a ceramic material to the surface of the medical device.

In one embodiment, the method further comprises generating a live image of the one or more detectable agents applied to the one or more target surfaces; and comparing the live image to a reference image for determining to what degree the implanted medical device is covered by endothelial tissue.

In one embodiment, the method further comprises transmitting the live image to a processor and obtaining the reference image from a memory device in communication with the processor prior to comparing the live image to the reference image.

In one embodiment, the method further comprises determining what percentage of the implanted medical device is not covered by endothelial tissue.

In one embodiment, the method further comprises determining what percentage of the implanted medical device is covered by endothelial tissue.

Another aspect of the present disclosure includes method of detecting healing adjacent to an implanted medical device, wherein the method first comprises applying a pre-treatment to at least a surface of the medical device prior to implantation, thereby defining a pre-treated surface. Then, the method includes applying one or more detectable agents to one or more target surfaces subsequent to implantation of the medical device. The one or more target surfaces selected from the group consisting of: (a) a portion of the pre-treated surface of the medical device that is not covered by endothelial tissue, and (b) a surface of a layer of endothelial tissue that is covering at least a portion of the implanted medical device. The method then includes detecting the one or more detectable agents applied to the one or more target surfaces for determining to what degree the implanted medical device is covered by endothelial tissue.

In one embodiment, applying the pre-treatment to the surface of the medical device prior to implantation comprises at least one of: (a) attaching a molecularly imprinted polymer to the surface of the medical device, (b) attaching a plurality of antibodies to the surface of the medical device, (c) forming at least one indentation on the surface of the medical device, (d) forming at least one protrusion on the surface of the medical device, (e) scratching the surface of the medical device, (f) applying a design to the surface of the medical device, (g) forming a pattern on the surface of the medical device, (h) attaching a plurality of dots to the surface of the medical device, (i) attaching a plurality of quantum dots to the surface of the medical device, (j) attaching a layer of a polymer material to the surface of the medical device, (k) attaching a layer of a metallic material to the surface of the medical device, (l) attaching a layer of a polymeric material to the surface of the medical device, and (m) attaching a layer of a ceramic material to the surface of the medical device.

In one embodiment, applying the one or more detectable agents comprises at least one of: (a) applying a paint to the surface of the medical device that is not covered by endothelial tissue, (b) applying a dye to the surface of the medical device that is not covered by endothelial tissue, (c) applying a magnetic field to the medical device and attaching a plurality of magnetic particles to the surface of the device that is not covered by endothelial tissue, (d) attaching a plurality of microbubbles to the surface of the medical device that is not covered by endothelial tissue, (e) attaching a plurality of colored microbubbles to the surface of the medical device that is not covered by endothelial tissue, (f) attaching a plurality of nanoparticles to the surface of the medical device that is not covered by endothelial tissue, (g) attaching a plurality of antigens to a plurality of antibodies connected to the surface of the medical device that is not covered by endothelial tissue, (h) attaching a plurality of molecules to a molecularly imprinted polymer connected to the surface of the medical device that is not covered by endothelial tissue, (i) applying a paint to the surface of the layer of endothelial tissue that is covering at least a portion of the medical device, (j) applying a dye to the surface of the layer of endothelial tissue that is covering at least a portion of the medical device, (k) attaching a plurality of microbubbles to the surface of the layer of endothelial tissue that is covering at least a portion of the medical device, (l) attaching a plurality of colored microbubbles to the surface of the layer of endothelial tissue that is covering at least a portion of the medical device, (m) attaching a plurality of microbubbles and liposomes to the surface of the layer of endothelial tissue that is covering at least a portion of the medical device, (n) attaching a plurality of microbubbles and liposomes modified with fluorescent agents to the surface of the layer of endothelial tissue that is covering at least a portion of the medical device, and (o) attaching a plurality of nanoparticles to the surface of the layer of endothelial tissue that is covering at least a portion of the medical device.

In one embodiment, the method further comprises generating a live image of the one or more detectable agents applied to the one or more target surfaces; and comparing the live image to a reference image for determining to what degree the implanted medical device is covered by endothelial tissue.

In one embodiment, the method further comprises transmitting the live image to a processor and obtaining the reference image from a memory device in communication with the processor prior to comparing the live image to the reference image.

In one embodiment, the method further comprises determining what percentage of the implanted medical device is not covered by endothelial tissue.

In one embodiment, the method further comprises determining what percentage of the implanted medical device is covered by endothelial tissue.

Another aspect of the present disclosure includes a method of detecting healing adjacent to an implanted medical device, wherein the method first comprises magnetizing the medical device subsequent to implantation. Then, the method comprises injecting a plurality of particles at a location adjacent to the medical device subsequent to magnetizing the medical device such that the particles attach to the medical device only at locations where the medical device is not covered by endothelial tissue. Then, the method comprises detecting the portions of the medical device that are not covered by endothelial tissue by detecting the particles.

Another aspect of the present disclosure includes a method of detecting healing adjacent to an implanted medical device, wherein the method first comprises attaching a molecularly imprinted polymer to a surface of the medical device prior to implantation. Then, the method comprises injecting a molecular agent at a location adjacent to the medical device subsequent to implantation such that the molecular agent binds to the molecularly imprinted polymer only at locations where the implanted medical device is not covered by endothelial tissue. Then, the method comprises detecting the portions of the medical device that are not covered by endothelial tissue by detecting the molecular agent.

Another aspect of the present disclosure includes a method of detecting healing adjacent to an implanted medical device, wherein the method first comprises attaching a plurality of antibodies to a surface of the medical device prior to implantation. Then, the method comprises injecting a plurality of antigens at a location adjacent to the medical device subsequent to implantation such that the plurality of antigens binds to the plurality of antibodies only at locations where the implanted medical device is not covered by endothelial tissue. Then, the method comprises detecting the portions of the medical device that are not covered by endothelial tissue by detecting the antigens.

Another aspect of the present disclosure includes a method of detecting healing adjacent to an implanted medical device, wherein the method first comprises injecting a plurality of microbubbles at a location adjacent to the medical device subsequent to implantation such that the plurality of microbubbles bind to the medical device only at locations where the implanted medical device is not covered by endothelial tissue. Then, the method comprises detecting the portions of the medical device that are not covered by endothelial tissue by detecting the microbubbles.

Another aspect of the present disclosure includes a method of detecting healing adjacent to an implanted medical device, wherein the method first comprises injecting a plurality of microbubbles at a location adjacent to the medical device subsequent to implantation such that the plurality of microbubbles bind to the endothelial tissue covering the implanted medical device. Then, the method comprises detecting the portions of the medical device that are covered by endothelial tissue by detecting the microbubbles.

Another aspect of the present disclosure includes a method of detecting healing adjacent to an implanted medical device, wherein the method first comprises injecting a first plurality of microbubbles at a location adjacent to the medical device subsequent to implantation such that the first plurality of microbubbles bind to the medical device only at locations where the implanted medical device is not covered by endothelial tissue. Then, the method comprises injecting a second plurality of microbubbles at a location adjacent to the medical device subsequent to implantation such that the second plurality of microbubbles bind to the endothelial tissue covering the implanted medical device. Then, the method comprises detecting the portions of the medical device that are covered by endothelial tissue by detecting the first and second pluralities of microbubbles.

A still further aspect of the present disclosure is directed to a system for detecting healing adjacent to an implanted medical device. The system includes a first catheter, a detection device, a memory, and a processor. The detection device is carried by the first catheter for delivery into the blood vessel. The detection device is adapted to capture a live image of at least one of (a) a portion of the implanted medical device that is not covered by endothelial tissue, and (b) a surface of a layer of endothelial tissue that is covering the implanted medical device. The memory stores one or more reference images of one or more reference medical devices. The processor is in communication with the detection device for comparing the live image generated by the detection device to at least one of the one or more reference images to determine the degree to which the implanted medical device is covered with endothelial tissue.

In one embodiment, the system further comprises a delivery device carried by the first catheter. The delivery device is for delivering a detectable agent to a location adjacent to the implanted medical device such that the detectable agent can attach to one or more of the implanted medical device and the endothelial tissue adjacent to the implanted medical device, the detectable agent for detection by the detection device.

In one embodiment, the delivery device comprises an injection needle.

In another embodiment, the system comprises a second catheter and a delivery device carried by the second catheter. The delivery device is for delivering a detectable agent to a location adjacent to the implanted medical device such that the detectable agent can attach to one or more of the implanted medical device and the endothelial tissue adjacent o the implanted medical device, the detectable agent for detection by the detection device.

In one embodiment, the delivery device comprises an injection needle.

In one embodiment, the detection device is selected from the group consisting of: (a) an optical coherence tomography device, (b) an ultrasound device, (c) a magnetic resonance imaging device, (d) an x-ray imaging device, (e) a fluorescence imaging device, (f) a near-infrared fluorescence (NIRF) imaging device, and (g) a camera.

### BRIEF DESCRIPTION OF THE DRAWINGS

FIG. 1 is a cut-away perspective view of a medical device constructed and implanted within an artery in accordance with the principles of the present disclosure;

FIG. 1A is a close-up view of the stent of FIG. 1 take from circle IA thereof;

FIG. 2 is a flowchart of one method for detecting healing adjacent to implanted medical device in accordance with the principles of the present disclosure;

FIG. 3 is a close-up view of an alternative stent taken from the circle IA of FIG. 1;

FIG. 3A is a cross-sectional view of one embodiment of the stent of FIG. 3 taken from line IIIA thereof;

FIG. 3B is a cross-sectional view of another embodiment of the stent of FIG. 3 taken through line IIIB thereof;

FIG. 4 is a flowchart of an alternative method for detecting healing adjacent to implanted medical device in accordance with the principles of the present disclosure; and

FIG. 5 is a schematic representation of system for detecting healing adjacent to implanted medical device in accordance with the principles of the present disclosure.

### DETAILED DESCRIPTION

Although the following text sets forth a detailed description of numerous different embodiments, it should be understood that the legal scope of the invention is defined by the words of the claims set forth at the end of this document. The detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims.

It should also be understood that, unless a term is expressly defined in this patent using the sentence “As used herein, the term ‘______’ is hereby defined to mean . . .” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. §112, sixth paragraph.

The present disclosure relates to a system and method for determining the degree to which healing has occurred adjacent to an implanted medical device. In one method, a surface of the medical device is pre-treated before implantation to increase its detectability after plantation. In another method, a surface of the medical device and/or a surface of any healed tissue covering the medical device is treated after implantation. Similar to the prior method, such treatment of the device and/or the healed tissue increases the detectability of the device and/or the healed tissue. So configured, a detection device is utilized to detect the treated surface(s) and generate information such as an image of the implanted device and/or the healed tissue. This information can be presented to a medical professional or a computer, for example, to determine the degree of healing adjacent to the medical device. Based on the degree of healing, the professional or the computer can then assess the existence of any risk of thrombosis or other healing-related concerns.

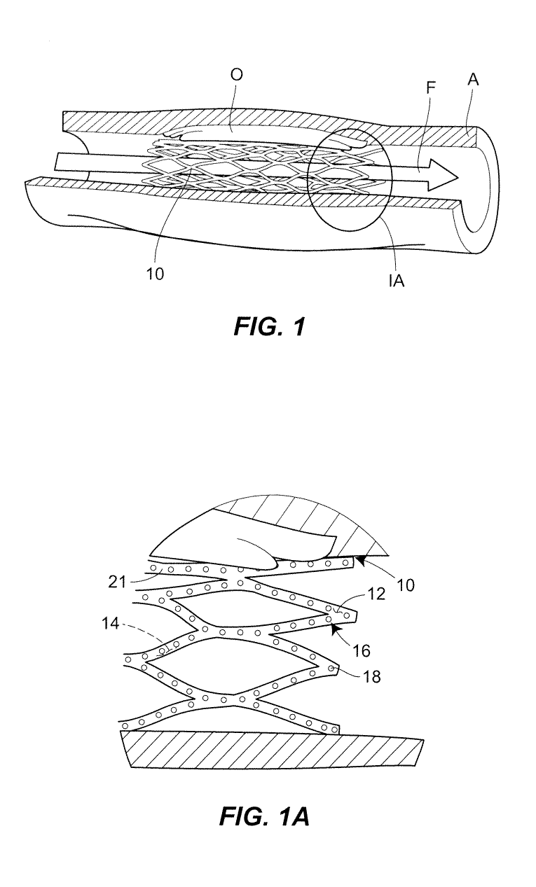

For the sake of description, the present disclosure describes a medical device that constitutes a mesh stent 10 adapted for implantation within a coronary artery A, as illustrated in FIGS. 1 and 1A. The stent 10 includes a mesh tube having an inner cylindrical surface 12 and an outer cylindrical surface 14. When implanted, the stent 10 expands such that the outer surface 14 compresses an obstruction O formed on the arterial wall. This compression at least partly removes the obstruction O from the flow path F of blood through the artery A, thereby ensuring proper blood flow. While the present description focuses on a stent 10, the principles set forth herein can apply to any medical device that is implanted within a patient, and which has blood contacting surfaces. For example, the principles could be applied to vascular grafts, ventricular assist devices, or any other foreseeable device that a person having ordinary skill in the art would determine is within the scope of the present disclosure.

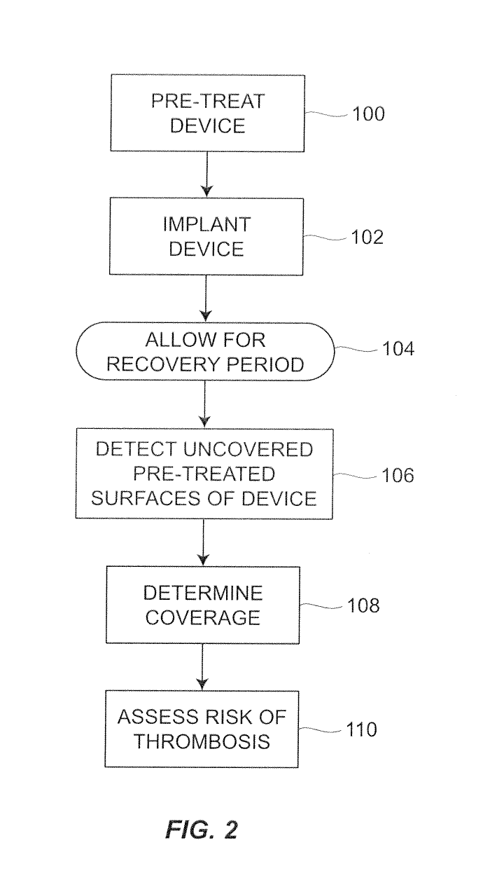

With reference now to FIG. 2, one method formulated in accordance with the principles of the present disclosure initially includes pre-treating the stent 10 (Block 100) prior to implantation. As will be described in more detail below, pre-treating the stent 10 can include applying a structural pre-treatment, a chemical or biological pre-treatment, or generally any other type of pre-treatment to at least the inner surface 12 (shown in FIG. 1A) of the stent 10 to increase its detectability.

After the stent 10 is pre-treated, a medical professional implants the stent 10 into the patient (Block 102) using a balloon catheter, for example. Once implanted, a pre-determined recovery period is allowed to lapse (Block 104), thereby providing the patient sufficient time to react to the presence of the implanted stent 10. A positive reaction will result in the generation of a layer of endothelial tissue that covers at least a portion of the inner surface 12 of the stent 10. Any portion of the inner surface 12 of the stent 10 that remains uncovered and in contact with blood can influence the risk of thrombosis. Thus, after the predetermined recovery period has lapsed, a procedure is undergone to detect the portion or portions of the inner surface 12 of the stent 10 that remains uncovered by endothelial tissue (Block 106). This detection provides information to the medical professional, for example, to determine the degree to which the stent 10 is covered (Block 108). This information may be conveyed to the medical professional in the folio of a digital image or a graph presented on a display device, or a set of data printed on a printout, for example, or in generally any other suitable manner. Based on this determination, the medical professional can then assess the patient's risk of thrombosis (Block 110).

Detecting the portion or portions of the surface 12 of the stent 10 that remain uncovered by endothelial tissue in accordance with Block 106 of FIG. 2 can be achieved using various different techniques and/or devices. One technique includes optical coherence tomography (OCT). In performing OCT, an OCT device could constitute a device positioned externally to the patient, or internally via an intravascular OCT device delivered by a catheter to the location of the stent 10. OCT devices emit light waves for eflection off of the implanted stent 10. The reflected light waves are then captured and an image of the stent 10 can be generated.

Other techniques available for detecting the inner surface 12 of the stent 10 can include the use of an ultrasound device, an intravascular ultrasound device, a magnetic resonance imaging (MRI) device, an intravascular magnetic resonance imaging device, an x-ray imaging device, an intravascular x-ray imaging device, a fluorescence imaging device, an intravascular fluorescence imaging device, a near-infrared fluorescence (NIRF) imaging device, an intravascular near-infrared fluorescence (NIRF) imaging device, a camera, or generally any other device capable of serving the intended purpose.

Referring back to FIG. 1, the stent 10 of the presently disclosed embodiment includes a wire mesh stent constructed of any known biocompatible material suitable for stent application. Some suitable materials can include stainless steel, gold, tantalum, a shape memory metal such as nickel-titanium alloy (e.g., nitinol), cobalt-chromium-nickel-molybdenum-iron alloy (ASTMF1058 and ISO 5832-7), shape memory polymers, and/or any other material suitable for the intended purpose. Accordingly, the stent 10 of the present disclosure is not limited to being constructed of any specific material.

As mentioned above with reference to FIG. 2, the pre-treatment of the stent 10 prior to implantation can comprise a structural pre-treatment, a chemical or biological pre-treatment, or any other e of pre-treatment suitable for increasing the detectability of the stent 10.

Generally, structural pre-treatments would be aimed at increasing the reflectivity of at least the inner surface 12 of the stent 10. So treated, the inner surface 12 would beneficially reflect a higher intensity signal when detection is undergone using OCT, ultrasound, fluorescence imaging, digital photography, or any other suitable technique that is based on capturing retlected signals.

As shown in FIG. 1A, structurally pre-treating the stent 10 to increase the reflectivity can include forming at least one indentation 16 on at least the inner surface 12 of the stent 10. The at least one indentation 16 of the form depicted includes a plurality of dimples 18 that could be formed by sandblasting or some other means. Additionally, the plurality of dimples 18 could be formed by submerging the stent 10 in a chemical bath that removes material from the stent 10 to create the dimples 18. Alternatively, structurally pre-treating the stent 10 could include forming at least one protrusion 21 on at least the inner surface 12 of the stent 10. The at least one protrusion 21 could be formed by removing material from the stent 10, similar to the method of forming the plurality of dimples 18, or could foreseeably include adding material to at least the inner surface 12 of the stent 10 by way of chemical vapor deposition or any other suitable means, for example.

Other methods of structurally treating the stent 10 could include scratching at least the inner surface 12 of the stent 10. The inner surface 12 of the stent 10 could be scratched by a wire brush, steel wool, or some other means. Still further, structurally treating the stent 10 could include applying a design to, or forming a pattern on, at least the inner surface 12 of the stent 10. The design or pattern could be applied using an adhesive, a weld bead, an ink, a dye, or any other suitable means. Still further, structurally treating the stent 10 could include attaching a plurality of dots such as quantum dots to at least the inner surface 12 of the stent 10. Further yet, structurally treating the stent 10 could include attaching a layer of a polymer material such as silicone, polyethylene, or polyurethane, a metallic material, a polymeric material, a ceramic material, or any other type of material to at least the inner surface 12 of the stent 10.

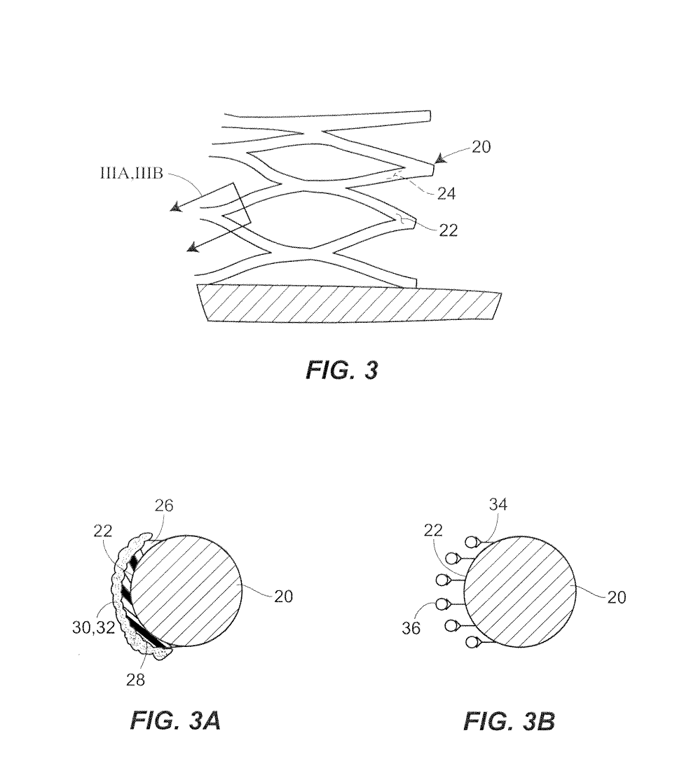

As mentioned, each of the foregoing methods of structurally pre-treating the stent 10 can be directed to increasing the reflectivity of the inner surface 12 of the stent 10, thereby increasing its detectability when using one or more of the available techniques discussed hereinabove. The present disclosure, however, is not limited to methods that include pre-treating a stent 10 or other medical device prior to implantation. For example, one alternative method can include treating a stent 20 having an inner surface 22 and an outer surface 24, as depicted in FIG. 3, after implantation.

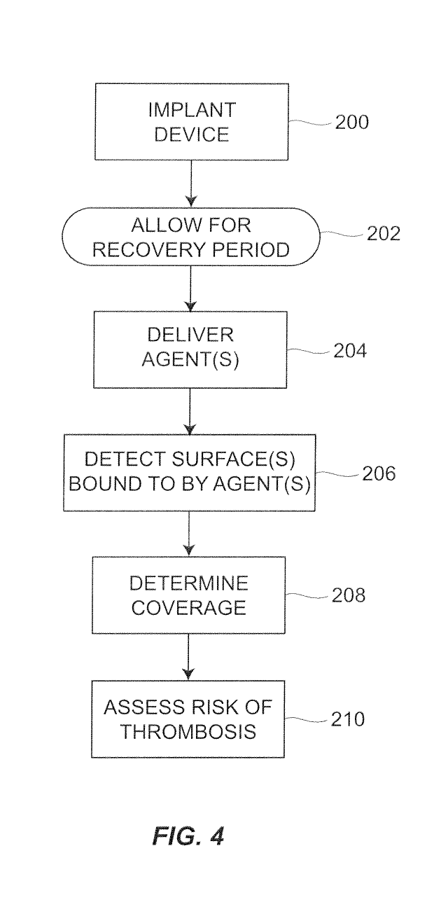

With reference to FIG. 4, this alternative method initially includes implanting the stent 20 (Block 200) into a patient using for example, a catheter-based delivery device. Once the stent 20 is implanted, a pre-deteimined recovery period is allowed to lapse (Block 202), thereby providing the patient sufficient time to react to the presence of the implanted stent 20. After the predetermined recovery period has lapsed, one or more detectable agents is delivered (Block 204) to the location of the stent 20. The one or more detectable agents are adapted or engineered to be highly detectable when using one or more of the detection methods/devices discussed above, or any other suitable detection method/device. Delivery of the one or more detectable agents can be achieved by way of introducing the agent or agents into the blood stream through a vein using a conventional hypodermic needle, or with a delivery device such as an injection needle attached to the end of a catheter that is positioned adjacent to the implanted stent 20.

In one embodiment, only a single detectable agent is delivered to the site of the stent 20. The single detectable agent either binds to the portions of the inner surface 22 of the stent 20 that remain uncovered by endothelial tissue, or to the surface of any layer of endothelial tissue that covers the stent 20. In an alternative embodiment, first and second detectable agents are delivered to the site of the stent 20. The first and second detectable agents can be different agents, and can be delivered at the same time or at different times. The first detectable agent binds to the portion of the inner surface 22 of the stent 20 that remains uncovered by endothelial tissue, while the second detectable agent binds to the surface of any layer of endothelial tissue covering the stent 20.

After the one or more detectable agents is delivered, a procedure is undergone to detect the surface(s) to which the agent(s) is/are bound (Block 206). This detection provides information to the medical professional, for example, to determine the degree to which the stent 20 is covered by endothelial tissue (Block 208). Based on this determination, the medical professional can then assess the risk of thrombosis (Block 210) in the patient.

In accordance with Block 204 of FIG. 4, delivering the one or more detectable agents can include, for example, one or more of the following: applying a paint to the portion of the inner surface 22 of the stent 20 that remains uncovered by endothelial tissue, applying a dye to the portion of the inner surface 22 of the stent 20 that remains uncovered by endothelial tissue, applying a magnetic field to the stent 20 and attaching a plurality of magnetic particles to the portion of the inner surface 22 of the stent 20 that remains uncovered by endothelial tissue, attaching a plurality of microbubbles to the portion of the inner surface 22 of the stent 20 that remains uncovered by endothelial tissue, attaching a plurality of colored microbubbles to the portion of the inner surface 22 of the stent 20 that remains uncovered by endothelial tissue, attaching a plurality of nanoparticles to the portion of the inner surface 22 of the stent 20 that remains uncovered by endothelial tissue, applying a paint to the surface of the layer of endothelial tissue covering at least a portion of the inner surface 22 of the stent 20, applying a dye to the surface of the layer of endothelial tissue covering at least a portion of the inner surface 22 of the stent 20, attaching a plurality of microbubbles to the surface of the layer of endothelial tissue covering at least a portion of the inner surface 22 of the stent 20, attaching a plurality of colored microbubbles to the surface of the layer of endothelial tissue covering at least a portion of the inner surface 22 of the stent 20, attaching a plurality of microbubbles and liposomes to the surface of the layer of endothelial tissue covering at least a portion of the inner surface 22 of the stent 20, attaching a plurality of microbubbles and liposomes modified with fluorescent agents to the surface of the layer of endothelial tissue covering at least a portion of the inner surface 22 of the stent 20, attaching a plurality of nanoparticles to the surface of the layer of endothelial tissue covering at least a portion of the inner surface 22 of the stent 20.

In view of the foregoing, it should be appreciated that when only a single detectable agent is delivered, the detection carried out in Block 206 of FIG. 4 can include detecting only the single surface to which the single detectable agent is bound. Alternatively, when first and second detectable agents are delivered, the detection carried out in Block 206 of FIG. 4 includes detecting the surfaces to which each of the first and second detectable agents is bound. In one embodiment, the first and second detectable agents are selected, designed, and/or engineered to be contrasting agents such that upon detection by one or more of the detection devices described herein, or otherwise, an image is generated that provides a stark contrast between the inner surface 22 of the stent 20 and the endothelial tissue.

In a variation on the method described with reference to FIG. 4, one method of detecting healing adjacent to an implanted medical device in accordance with the principles of the present disclosure can include both pre-treating the stent 20 prior to implantation and treating the stent 20 after implantation. For example, one such embodiment can include applying a chemical or biological pre-treatment layer to the stent 20 and a corresponding chemical or biological detectable agent after implantation.

For example, with reference to FIG. 3A, in one embodiment, the chemical or biological pre-treatment layer can include a coating or layer of a molecularly imprinted polymer 26 attached to at least the inner surface 22 of the stent 20. Such a molecularly imprinted polymer 26 contains a plurality of recesses 28 adapted to receive the molecules 30 with which the polymer 26 was imprinted, thereby forming a molecular layer 32 on top of the polymer 26. In accordance with the present disclosure, the molecular layer 32 would include molecules 30 that are highly detectable using any one of the detection methods/devices described above, or otherwise. Accordingly, in this embodiment, delivering the detectable agent in accordance with Block 204 of FIG. 4 would include delivering a plurality of the specific molecules 30. The molecules 30 would be attracted to the molecularly imprinted polymer 26 such that they bind to the recesses 28 disposed along the portion of the inner surface 22 of the stent 20 that remains uncovered by endothelial tissue. So configured, the molecules 30 provide a highly detectable agent for assisting in the detection of the uncovered portions of the stent 20.

Referring to FIG. 3B, another embodiment involving a chemical or biological pre-treatment could include attaching a plurality of antibodies 34 to at least the inner surface 22 of the stent 20 prior to implantation. Then, subsequent to implantation and in accordance with Block 204 of FIG. 4, a plurality of antigens 36 could be delivered to serve as the detectable agent. Upon delivery, the antigens 36 would bind to the antibodies 34 disposed along the portion of the inner surface 22 of the stent 20 that remains uncovered by endothelial tissue. The antigens 36 would be selected or engineered to specifically provide a high level of detectability when using any one of the detection methods/devices described above, or otherwise. So configured, the antigens 36 would assist in the detection of the uncovered portions of the stent 20, thereby serving as the detectable agent in the method described with reference to FIG. 4.

While the present disclosure has thus far described a system and method for detecting healing adjacent to an implanted medical device wherein a medical professional determines the degree of coverage of endothelial tissue and any associated risk of thrombosis based on information provided by a detection device, an alternative embodiment can foreseeably perform either or both of these determinations automatically.

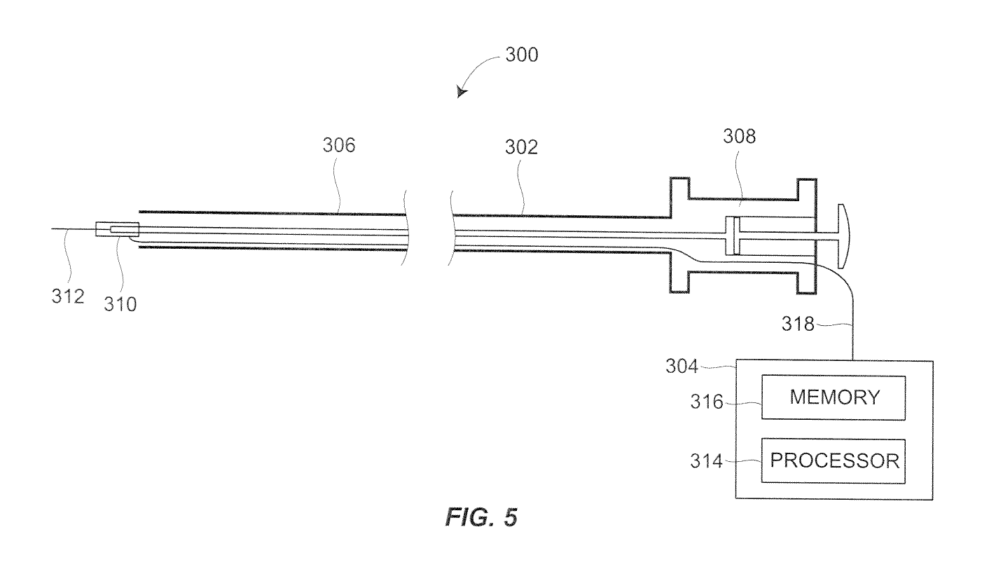

For example, FIG. 5 depicts a system 300 for detecting healing adjacent to an implanted medical device constructed in accordance with the present disclosure. The system 300 generally includes a catheter 302 and a computer 304. The catheter 302 includes an elongated sheath 306, a handle 308, a detection device 310, and a delivery device 312. The detection device 310 is attached to an end of the sheath 306 opposite the handle 308. The handle 308 is for manipulating the sheath 306 through a blood vessel of the patient to position the detection device 310 adjacent to the implanted medical device 10, 20. The detection device 310 is adapted to detect any portion of the implanted medical device 10, 20 that is not covered by endothelial tissue and/or any layer of endothelial tissue covering portions of the device 10, 20, as described in the various methods provided above. The information detected by the detection device 310, which can consist of a live image such as a digital image of the implanted medical device 10, 20 and/or adjacent endothelial tissue, is then transmitted to the computer 304 via a cable 318 for processing. Alternatively, this transmission could be accomplished via wireless communication between the detection device 310 and the computer 304.

In the disclosed embodiment, the computer 304 includes a processor 314 and a memory 316. The memory 316 can store one or more reference images of various medical devices used in conjunction with the system 300. For example, if the system is used to detect the healing adjacent to five different types and/or sizes of medical stents, the memory 314 would include a reference image of each stent. The system 300 and the memory 314 are not limited to applications involving stents and, as such, the reference images could include any number of reference images of any number of medical devices. Upon receiving the information from the detection device 310, the processor 316 retrieves the appropriate reference image from the memory 314 and compares the reference image to the live image transmitted by the detection device 310. Based on this comparison, the processor 316 determines to what degree the stent 10, 20 is covered by endothelial tissue and presents this information to the medical professional. In one embodiment, this information may be presented in the form of a percentage, for example. In another embodiment, the processor can be pre-programmed to determine not only the degree to which the stent 10, 20 is covered by endothelial tissue, but also any corresponding risk of thrombosis. In this arrangement, the computer 304 may transmit either or both pieces of information to the medical professional.

As mentioned, the system 300 depicted in FIG. 5 further includes the delivery device 312, which includes an injection needle delivery device. The delivery device 312 is carried by the catheter for delivering the one or more detectable agents to a location adjacent to the implanted stent 20, in accordance with Block 204 of FIG. 4. This configuration advantageously combines the delivery device 312 and the detection device 310 into a single arrangement such as to minimize the number of catheters that must be delivered and removed from the patient during a procedure that includes the method described with reference to FIG. 4. In alternative embodiments of the system 300, the detection device 310 and delivery device 312 can be carried by different catheters. Finally, as should be appreciated in view of the foregoing description, the detection device 310 of the system 300 depicted in FIG. 5 can include any one or more of the following: an optical coherence tomography device, an ultrasound device, a magnetic resonance imaging device, an x-ray imaging device, a fluorescence imaging device, a near-infrared fluorescence (NIRF) imaging device, a camera, or any other device capable of providing the desired functionality.

In view of the foregoing, it should be appreciated that the present disclosure is not limited to, or defined by, the embodiments described in the specification. These are merely examples of what is intended to be the entire scope of the present application.

1. A method of detecting healing adjacent to an implanted medical device, the method comprising:
pre-treating a surface of the medical device prior to implantation, thereby defining a pre-treated surface of the medical device; and
detecting a portion of the pre-treated surface of the medical device subsequent to implantation, the portion of the pre-treated surface not being covered by endothelial tissue.

2. The method of claim 1, wherein detecting the portion of the pre-treated surface of the medical device comprises delivering a detection device into the blood vessel to a position adjacent the implanted medical device.

3. The method of claim 1, wherein detecting the portion of the pre-treated surface of the medical device comprises detecting the pre-treated surface with at least one of:
(a) an optical coherence tomography device,
(b) an intravascular optical coherence tomography device,
(c) an ultrasound device,
(d) an intravascular ultrasound device,
(e) a magnetic resonance imaging device,
(f) an intravascular magnetic resonance imaging device,
(g) an x-ray imaging device,
(h) an intravascular x-ray imaging device,
(i) a fluorescence imaging device,
(j) an intravascular fluorescence imaging device,
(k) a near-infrared fluorescence (NIRF) imaging device,
(l) an intravascular near-infrared fluorescence (NIRF) imaging device, and
(m) a camera.

4. The method of claim 1, wherein pre-treating the surface of the medical device comprises structurally treating the surface of the medical device.

5. The method of claim 4, wherein structurally treating the surface of the medical device comprises at least one of:
(a) forming at least one indentation on the surface of the medical device,
(b) forming at least one protrusion on the surface of the medical device,
(c) scratching the surface of the medical device,
(d) applying a design to the surface of the medical device,
(e) forming a pattern on the surface of the medical device,
(f) attaching a plurality of dots to the surface of the medical device,
(g) attaching a plurality of quantum dots to the surface of the medical device,
(h) attaching a layer of a polymer material to the surface of the medical device,
(i) attaching a layer of a metallic material to the surface of the medical device,
(j) attaching a layer of a polymeric material to the surface of the medical device, and
(k) attaching a layer of a ceramic material to the surface of the medical device.

6. The method of claim 1, further comprising:
generating a live image of at least the pre-treated surface of the medical device that is not covered by endothelial tissue;
comparing the live image to a reference image for determining to what degree the implanted medical device is covered by endothelial tissue.

7. The method of claim 6, further comprising transmitting the live image to a processor and obtaining the reference image from a memory device in communication with the processor prior to comparing the live image to the reference image.

8. The method of claim 1, further comprising determining what percentage of the implanted medical device is not covered by endothelial tissue.

9. The method of claim 1, further comprising determining what percentage of the implanted medical device is covered by endothelial tissue.

10. A method of detecting healing adjacent to an implanted medical device, the method comprising:
applying one or more detectable agents to one or more target surfaces subsequent to implantation of the medical device, the one or more target surfaces selected from the group consisting of:
a surface of the medical device that is not covered by endothelial tissue, and
a surface of a layer of endothelial tissue, the layer covering at least a portion of the medical device; and
detecting the one or more detectable agents applied to the one or more target surfaces for determining to what degree the implanted medical device is covered by endothelial tissue.

11. The method of claim 10, wherein applying the one or more detectable agents comprises at least one of:
(a) applying a paint to the surface of the device that is not covered by endothelial tissue,
(b) applying a dye to the surface of the medical device that is not covered by endothelial tissue,
(c) applying a magnetic field to the medical device and attaching a plurality of magnetic particles to the surface of the device that is not covered by endothelial tissue,
(d) attaching a plurality of microbubbles to the surface of the medical device that is not covered by endothelial tissue,
(e) attaching a plurality of colored microbubbles to the surface of the medical device that is not covered by endothelial tissue,
(f) attaching a plurality of nanoparticles to the surface of the medical device that is not covered by endothelial tissue,
(g) attaching a plurality of antigens to a plurality of antibodies connected to the surface of the medical device that is not covered by endothelial tissue,
(h) attaching a plurality of molecules to a molecularly imprinted polymer connected to the surface of the medical device that is not covered by endothelial tissue,
(i) applying a paint to the surface of the layer of endothelial tissue that is covering at least a portion of the medical device,
(j) applying a dye to the surface of the layer of endothelial tissue that is covering at least a portion of the medical device,
(k) attaching a plurality of microbubbles to the surface of the layer of endothelial tissue that is covering at least a portion of the medical device,
(l) attaching a plurality of colored microbubbles to the surface of the layer of endothelial tissue that is covering at least a portion of the medical device,
(m) attaching a plurality of microbubbles and liposomes to the surface of the layer of endothelial tissue that is covering at least a portion of the medical device,
(n) attaching a plurality of microbubbles and liposomes modified with fluorescent agents to the surface of the layer of endothelial tissue that is covering at least a portion of the medical device, and
(o) attaching a plurality of nanoparticles to the surface of the layer of endothelial tissue that is covering at least a portion of the medical device.

12. The method of claim 10, further comprising pre-treating a surface of the medical device prior to implantation, thereby defining a pre-treated surface of the medical device, such that applying the one or more detectable agents to the surface of the medical device that is not covered by endothelial tissue comprises applying one or more detectable agents to the pre-treated surface.

13. The method of claim 12, wherein pre-treating the surface of the medical device prior to implantation comprises at least one of:
(a) attaching a molecularly imprinted polymer to the surface of the medical device,
(b) attaching a plurality of antibodies to the surface of the medical device,
(c) forming at least one indentation on the surface of the medical device,
(d) forming at least one protrusion on the surface of the medical device,
(e) scratching the surface of the medical device,
(f) applying a design to the surface of the medical device,
(g) forming a pattern on the surface of the medical device,
(h) attaching a plurality of dots to the surface of the medical device,
(i) attaching a plurality of quantum dots to the surface of the medical device,
(j) attaching a layer of a polymer material to the surface of the medical device,
(k) attaching a layer of a metallic material to the surface of the medical device,
(l) attaching a layer of a polymeric material to the surface of the medical device, and
(m) attaching a layer of a ceramic material to the surface of the medical device.

14. The method of claim 10, further comprising:
generating a live image of the one or more detectable agents applied to the one or more target surfaces; and
comparing the live image to a reference image for determining to what degree the implanted medical device is covered by endothelial tissue.

15. The method of claim 14, further comprising transmitting the live image to a processor and obtaining the reference image from a memory device in communication with the processor prior to comparing the live image to the reference image.

16. The method of claim 10, further comprising determining what percentage of the implanted medical device is not covered by endothelial tissue.

17. The method of claim 10, further comprising determining what percentage of the implanted medical device is covered by endothelial tissue.

18. A method of detecting healing adjacent to an implanted medical device, the method comprising:
applying a pre-treatment to at least a surface of the medical device prior to implantation, thereby defining a pre-treated surface;
applying one or more detectable agents to one or more target surfaces subsequent to implantation of the medical device, the one or more target surfaces selected from the group consisting of:
a portion of the pre-treated surface of the medical device that is not covered by endothelial tissue, and
a surface of a layer of endothelial tissue that is covering at least a portion of the implanted medical device; and
detecting the one or more detectable agents applied to the one or more target surfaces for determining to what degree the implanted medical device is covered by endothelial tissue.

19. The method of claim 18, wherein applying the pre-treatment to the surface of the medical device prior to implantation comprises at least one of:
(a) attaching a molecularly imprinted polymer to the surface of the medical device,
(b) attaching a plurality of antibodies to the surface of the medical device,
(c) forming at least one indentation on the surface of the medical device,
(d) forming at least one protrusion on the surface of the medical device,
(e) scratching the surface of the medical device,
(f) applying a design to the surface of the medical device,
(g) forming a pattern on the surface of the medical device,
(h) attaching a plurality of dots to the surface of the medical device,
(i) attaching a plurality of quantum dots to the surface of the medical device,
(j) attaching a layer of a polymer material to the surface of the medical device,
(k) attaching a layer of a metallic material to the surface of the medical device,
(l) attaching a layer of a polymeric material to the surface of the medical device, and
(m) attaching a layer of a ceramic material to the surface of the medical device.

20. The method of claim 18, wherein applying the one or more detectable agents comprises at least one of:
(a) applying a paint to the surface of the medical device that is not covered by endothelial tissue,
(b) applying a dye to the surface of the medical device that is not covered by endothelial tissue,
(c) applying a magnetic field to the medical device and attaching a plurality of magnetic particles to the surface of the device that is not covered by endothelial tissue,
(d) attaching a plurality of microbubbles to the surface of the medical device that is not covered by endothelial tissue,
(e) attaching a plurality of colored microbubbles to the surface of the medical device that is not covered by endothelial tissue,
(f) attaching a plurality of nanoparticles to the surface of the medical device that is not covered by endothelial tissue,
(g) attaching a plurality of antigens to a plurality of antibodies connected to the surface of the medical device that is not covered by endothelial tissue,
(h) attaching a plurality of molecules to a molecularly imprinted polymer connected to the surface of the medical device that is not covered by endothelial tissue,
(i) applying a paint to the surface of the layer of endothelial tissue that is covering at least a portion of the medical device,
(j) applying a dye to the surface of the layer of endothelial tissue that is covering at least a portion of the medical device,
(k) attaching a plurality of microbubbles to the surface of the layer of endothelial tissue that is covering at least a portion of the medical device,
(l) attaching a plurality of colored microbubbles to the surface of the layer of endothelial tissue that is covering at least a portion of the medical device,
(m) attaching a plurality of microbubbles and liposomes to the surface of the layer of endothelial tissue that is covering at least a portion of the medical device,
(n) attaching a plurality of microbubbles and liposomes modified with fluorescent agents to the surface of the layer of endothelial tissue that is covering at least a portion of the medical device, and
(o) attaching a plurality of nanoparticles to the surface of the layer of endothelial tissue that is covering at least a portion of the medical device.

21. The method of claim 18, further comprising:
generating a live image of the one or more detectable agents applied to the one or more target surfaces; and
comparing the live image to a reference image for determining to what degree the implanted medical device is covered by endothelial tissue.

22. The method of claim 21, further comprising transmitting the live image to a processor and obtaining the reference image from a memory device in communication with the processor prior to comparing the live image to the reference image.

23. The method of claim 18, further comprising determining what percentage of the implanted medical device is not covered by endothelial tissue.

24. The method of claim 18, further comprising determining what percentage of the implanted medical device is covered by endothelial tissue.

25. A method of detecting healing adjacent to an implanted medical device, the method comprising:
magnetizing the medical device subsequent to implantation;
injecting a plurality of particles at a location adjacent to the medical device subsequent to magnetizing the medical device such that the particles attach to the medical device only at locations where the medical device is not covered by endothelial tissue;
detecting the portions of the medical device that are not covered by endothelial tissue by detecting the particles.

26. A method of detecting healing adjacent to an implanted medical device, the method comprising:
attaching a molecularly imprinted polymer to a surface of the medical device prior to implantation;
injecting a molecular agent at a location adjacent to the medical device subsequent to implantation such that the molecular agent binds to the molecularly imprinted polymer only at locations where the implanted medical device is not covered by endothelial tissue;
detecting the portions of the medical device that are not covered by endothelial tissue by detecting the molecular agent.

27. A method of detecting healing adjacent to an implanted medical device, the method comprising:
attaching a plurality of antibodies to a surface of the medical device prior to implantation;
injecting a plurality of antigens at a location adjacent to the medical device subsequent to implantation such that the plurality of antigens bins to the plurality of antibodies only at locations where the implanted medical device is not covered by endothelial tissue;
detecting the portions of the medical device that are not covered by endothelial tissue by detecting the antigens.

28. A method of detecting healing adjacent to an implanted medical device, the method comprising:
injecting a plurality of microbubbles at a location adjacent to the medical device subsequent to implantation such that the plurality of microbubbles bind to the medical device only at locations where the implanted medical device is not covered by endothelial tissue;
detecting the portions of the medical device that are not covered by endothelial tissue by detecting the microbubbles.

29. A method of detecting healing adjacent to an implanted medical device, the method comprising:
injecting a plurality of microbubbles at a location adjacent to the medical device subsequent to implantation such that the plurality of microbubbles bind to the endothelial tissue covering the implanted medical device;
detecting the portions of the medical device that are covered by endothelial tissue by detecting the microbubbles.

30. A method of detecting healing adjacent to an implanted medical device, the method comprising:
injecting a first plurality of microbubbles at a location adjacent to the medical device subsequent to implantation such that the first plurality of microbubbles bind to the medical device only at locations where the implanted medical device is not covered by endothelial tissue;
injecting a second plurality of microbubbles at a location adjacent to the medical device subsequent to implantation such that the second plurality of microbubbles bind to the endothelial tissue covering the implanted medical device;
detecting the portions of the medical device that are covered by endothelial tissue by detecting the first and second pluralities of microbubbles.

31. A system for detecting healing adjacent to an implanted medical device, the system comprising:
a first catheter;
a detection device carried by the first catheter for delivery into the blood vessel, the detection device adapted to capture a live image of at least one of (a) a portion of the implanted medical device that is not covered by endothelial tissue, and (b) a surface of a layer of endothelial tissue that is covering the implanted medical device;
a memory storing one or more reference images of one or more reference medical devices; and
a processor in communication with the detection device for comparing the live image generated by the detection device to at least one of the one or more reference images to determine the degree to which the implanted medical device is covered with endothelial tissue.

32. The system of claim 31, further comprising:
a delivery device carried by the first catheter, the delivery device for delivering a detectable agent to a location adjacent to the implanted medical device such that the detectable agent can attach to one or more of the implanted medical device and the endothelial tissue adjacent to the implanted medical device, the detectable agent for detection by the detection device.

33. The system of claim 32, wherein the delivery device comprises an injection needle.

34. The system of claim 31, further comprising:
a second catheter; and
a delivery device carried by the second catheter, the delivery device for delivering a detectable agent to a location adjacent to the implanted medical device such that the detectable agent can attach to one or more of the implanted medical device and the endothelial tissue adjacent to the implanted medical device, the detectable agent for detection by the detection device.

35. The system of claim 34, wherein the delivery device comprises an injection needle.

36. The system of claim 31, wherein the detection device is selected from the group consisting of:
(a) an optical coherence tomography device,
(b) an ultrasound device,
(c) a magnetic resonance imaging device,
(d) an x-ray imaging device,
(e) a fluorescence imaging device,
(f) a near-infrared fluorescence (NIRF) imaging device, and
(g) a camera.
